(19) United States
(12) Patent Application Publication
(10) Pub. No.: US 2006/0036086 A1
(43) Pub. Date: Feb. 16, 2006

(54) Use of signal transduction inhibitors and combination therapies for the prevention or treatment of cancer and angiogenesis related diseases

(76) Inventors: Rakesh K. Jain, Boston, MA (US); Yotaro Izumi, Tokyo (JP); Lei Xu, Quincy, MA (US); Dai Fukumura, Newton, MA (US)

Correspondence Address: CLARK & ELBING LLP, 101 FEDERAL STREET, BOSTON, MA, 02110, US

(21) Appl. No.: 10/507,352

(22) PCT Filed: Mar. 5, 2003
(86) PCT No.: PCT/US03/06796

§ 371 Date: Aug. 17, 2005

Related U.S. Application Data
(63) Continuation-in-part of application No. 60/363,760, filed on Mar. 12, 2002.
(63) Continuation-in-part of application No. 60/366,017, filed on Mar. 20, 2002.
(60) Provisional application No. 60/363,760, filed on Mar. 12, 2002.
(60) Provisional application No. 60/366,017, filed on Mar. 20, 2002.

(51) Int. Cl. C12Q 1/68; C07H 21/02

(52) U.S. Cl. 536/23.1; 435/6

(57) ABSTRACT

The invention provides improved compositions (e.g., combinations of signal transduction inhibitors) and methods for the prevention, stabilization, or treatment of cancer or other angiogenesis related diseases. In particular, the present methods use combination therapies to modulate the expression or activity of multiple mRNA molecules or proteins associated with angiogenesis or cancer in a mammal (e.g., a human)

### STATEMENT AS TO FEDERALLY SPONSORED RESEARCH

This invention was funded by grant PO1-CA-80124-02 from the National Institutes of Health. The government may have certain rights in the invention.

### BACKGROUND OF THE INVENTION

In general, the invention features methods for the selection of a preferred therapy (e.g., one or more signal transduction inhibitors) for the treatment of a particular cancer patient or group of cancer patients. The invention also provides improved methods for the treatment and prevention of a variety of cancers and angiogenesis related diseases in mammals (e.g., humans).

Cancer is one of the leading causes of death. Some cancers respond poorly to chemotherapy or have initial favorable responses to chemotherapy but later develop resistance after repeated chemotherapy treatments. For example, some tumors survive anti-angiogenic therapy targeted against a single angiogenic factor, such as vascular endothelial growth factor (VEGF), by switching their dependence to other factors. In addition, multidrug-resistance genes found in some cancer cells enable the cells to pump out drugs, rendering the cancers resistant to multiple classes of drugs. Many of the current treatments that destroy cancerous cells also affect normal cells, resulting in a variety of possible side-effects, such as nausea, vomiting, low blood cell counts, increased risk of infection, hair loss, and ulcers in mucous membranes. In addition to cancer, there are a variety of angiogenesis related diseases that are associated with excessive or insufficient vascular growth.

Thus, improved therapies are needed that result in a higher incidence of remissions and longer lengths of remissions. Other desirable therapies prevent the initial occurrence of a cancer or another angiogenesis related disease or prevent the recurrence of a cancer or angiogenesis related disease. Preferably, the therapies produce few adverse side-effects and are useful for the treatment of a variety of cancers or angiogenesis related diseases.

### SUMMARY OF THE INVENTION

The purpose of the present invention is to provide improved methods for treating and preventing cancer and angiogenesis related diseases. In particular, these methods involve the selection of a preferred signal transduction inhibitor or a preferred combination therapy (e.g., a combination of signal transduction inhibitors) for a patient diagnosed with, or at increased risk for, a cancer or an angiogenesis related disease based on the patient's expression profile of cancer or angiogenesis related genes, such as pro-angiogenic or anti-angiogenic genes. Preferably, the therapy modulates the expression of multiple genes (e.g., 5, 10, 15, or more cancer or angiogenesis related genes) in the subject in an amount sufficient to prevent, stabilize, or treat cancer or an angiogenesis related disease.

Accordingly, in a first aspect, the invention provides a method of selecting a combination therapy for the treatment, stabilization, or prevention of a cancer or an angiogenesis related disease in a mammal. This method involves analyzing the expression profile of more than one mRNA and/or protein in a sample obtained from the mammal. A therapy is selected that includes two or more compounds (e.g., signal transduction inhibitors) that each (i) decrease the expression level or activity of an mRNA or protein that has a higher than normal expression level in the mammal and/or (ii) increase the expression level or activity of an mRNA or protein that has a lower than normal expression level in the mammal.

In a related aspect, the invention provides a method of selecting a signal transduction inhibitor for the treatment, stabilization, or prevention of a cancer or an angiogenesis related disease in a mammal. This method involves analyzing the expression profile of one or more mRNA molecules and/or proteins in a sample obtained from the mammal. A signal transduction inhibitor is selected that (i) decreases the expression level or activity of an mRNA or protein that has a higher than normal expression level in the mammal and/or (ii) increases the expression level or activity of an mRNA or protein that has a lower than normal expression level in the mammal.

In another related aspect, the invention provides a method for preventing, delaying, or treating a cancer or an angiogenesis related disease in a mammal. This method involves analyzing the expression profile of more than one mRNA and/or protein in a sample obtained from the mammal. A therapy is selected that includes two or more compounds (e.g., signal transduction inhibitors) that each (i) decrease the expression level or activity of an mRNA or protein that has a higher than normal expression level in the mammal and/or (ii) increase the expression level or activity of an mRNA or protein that has a lower than normal expression level in the mammal. The selected therapy is administered to the mammal in an amount sufficient to treat, stabilize, or prevent the cancer or angiogenesis related disease.

In yet another related aspect, the invention provides another method for preventing, delaying, or treating a cancer or an angiogenesis related disease in a mammal. This method involves analyzing the expression profile of one or more mRNA molecules and/or proteins in a sample obtained from the mammal. A signal transduction inhibitor is selected that (i) decreases the expression level or activity of an mRNA or protein that has a higher than normal expression level in the mammal and/or (ii) increases the expression level or activity of an mRNA or protein that has a lower than normal expression level in the mammal. The selected signal transduction inhibitor is administered to the mammal in an amount sufficient to treat, stabilize, or prevent the cancer or angiogenesis related disease.

The invention also provides methods for classifying subjects involved in a clinical trial for a combination chemotherapeutic or angiogenesis modulating therapy (e.g., a combination of signal transduction inhibitors) based on the subjects' expression profiles. This method allows the subjects who are most likely to benefit from a particular combination therapy to be included in the corresponding subgroup for the clinical trial. Thus, this method enables the association of a particular expression profile with improved drug efficacy to be demonstrated or confirmed in humans.

According to this aspect of the invention, a method is provided for stratification of subjects involved in a clinical trial of a combination therapy that includes two or more compounds (e.g., signal transduction inhibitors) for the treatment, stabilization, or prevention of a cancer or an angiogenesis related disease in a mammal. This method involves analyzing the expression profile of a sample obtained from a subject and determining the presence of a lower or higher than normal expression level for more than one mRNA and/or protein in the sample before, during, or after the clinical trial. The presence of a particular expression profile in the subject places the subject into a subgroup for the clinical trial or excludes the subject from a subgroup for the clinical trial.

In a related aspect, the invention provides a method for stratification of subjects involved in a clinical trial of a signal transduction inhibitor for the treatment, stabilization, or prevention of a cancer or an angiogenesis related disease in a mammal. This method involves analyzing the expression profile of a sample obtained from a subject and determining the presence of a lower or higher than normal expression level for one or more mRNA molecules and/or proteins in the sample before, during, or after the clinical trial. The presence of a particular expression profile in the subject places the subject into a subgroup for the clinical trial or excludes the subject from a subgroup for the clinical trial.

The invention also features combination therapies that are useful for the prevention, stabilization, or treatment of cancer or angiogenesis related diseases. Because these therapies contain multiple pharmaceutically active compounds, the therapies modulate the expression or activity of numerous cancer or angiogenesis related molecules.

In one such aspect, the invention features a pharmaceutical composition that includes at least 2, 3, 5, 7, 10, 15, or more pharmaceutically active compounds (e.g., signal transduction inhibitors) that each modulate the expression or activity of at least 1, 3, 5, 10, 15, 20, 30 or more cancer or angiogenesis related molecules (e.g., mRNA molecules or proteins). Preferably, the composition contains a pharmaceutically acceptable carrier. Suitable carriers include, but are not limited to, saline, buffered saline, dextrose, water, glycerol, ethanol, and combinations thereof. The composition can be adapted for the mode of administration and can be in the form of, for example, a pill, tablet, capsule, spray, powder, or liquid.

The invention also features a variety of databases. These databases include information on the effect of a compound on the expression or activity of cancer or angiogenesis related mRNA molecules or proteins. These databases may also be used in the development of combination therapies and in the selection of a preferred therapy for a particular patient or class of patients.

In one such aspect, the invention features an electronic database including at least 5, 10, 10², 10³, 10⁴, 10⁵, 10⁶, 10⁷, 10⁸, or 10⁹ records of mRNA molecules or proteins (e.g., cancer or angiogenesis related molecules) correlated to records of compounds (e.g., signal transduction inhibitors) and their ability to modulate the expression or activity of mRNA molecules or proteins. Preferably, the database includes records for at least 5, 10, 10², 10³, 10⁴, 10⁵, or 10⁶ compounds. In yet other embodiments, the database includes records for at least one protein expressed by an open reading frame for at least 0.5, 1, 5, 10, 20, 30, 40, 50, 60, 70, 80, 90, or 100% of the open reading frames in the genome of a mammal, such as a human.

In another aspect, the invention features a computer including a database of the invention and a user interface (i) capable of displaying one or more compounds (e.g., signal transduction inhibitors) that modulate the activity of an mRNA or protein whose record is stored in the computer or (ii) capable of displaying one or more mRNA molecules or proteins whose expression or activity is modulated by a compound whose record is stored in the computer. The internal components of the computer typically include a processor coupled to a memory. The external components usually include a mass-storage device, e.g., a hard disk drive; user input devices, e.g., a keyboard and a mouse; a display, e.g., a monitor; and optionally, a network link capable of connecting the computer system to other computers to allow sharing of data and processing tasks. Programs may be loaded into the memory of this system during operation.

In various embodiments of any of the aspects of the invention, the analysis of an expression profile includes comparing the expression level of an mRNA or protein in the sample to the corresponding level in a control sample (e.g. a sample from a healthy patient). In other embodiments, the expression level of an mRNA or protein in a sample from cancerous or diseased cells in the mammal is compared to the corresponding level in a sample from healthy cells in the mammal. In yet other embodiments, the expression profile includes the expression level of pro-angiogenic mRNA molecules or proteins, such as vascular endothelial growth factor, transforming growth factor alpha, angiopoietin-1, plasminogen activator inhibitor-1, or anti-angiogenic mRNA molecules or proteins, such as thrombospondin-1. Other exemplary angiogenesis related molecules are listed in FIGS. 1G and 3. Preferably, the expression profiling is performed using a DNA chip.

In certain embodiments, the expression profiling also includes the detection of the presence or absence of polymorphic or mutant forms of mRNA molecules or proteins. In preferred embodiments, the expression or activity of an mRNA or protein that has a mutation associated with cancer or an angiogenesis related disease and that has a higher than normal, normal, or even lower than normal level of expression is inhibited by a therapy of the invention. For subjects that express both a mutant form (e.g., a cancer-related form) and a wild-type form (e.g., a form not associated with cancer) of an mRNA or protein, the therapy preferably inhibits the expression or activity of the mutant form by at least 2, 5, 10, or 20-fold more than it inhibits the expression or activity of the wild-type form.

In preferred embodiments, at least 1, 2, 3, 5, 10, or all of the compounds in the combination therapy each modulate the expression or activity of at least 2, 3, 5, 10, 15, 20, 30, or more mRNA molecules or proteins. In other preferred embodiments, the therapy modulates the expression or activity of at least 2, 3, 5, 10, 15, 20, 30, or more mRNA molecules or proteins. In some embodiments, a compound or therapy increases the expression or activity of at least 2, 3, 5, 10, 15, 20, 30, or more anti-angiogenic or cancer suppressor mRNA molecules or proteins and decreases the expression or activity of at least 2, 3, 5, 10, 15, 20, 30, or more pro-angiogenic or oncogenic mRNA molecules or proteins. Preferably, the therapy modulates the expression or activity of at least 2, 3, 5, 10, 15, 20, 30, or more mRNA molecules or proteins by at least 20, 40, 50, 75, 90, 100, 200, 500, or even 1000%. In other preferred embodiments, at least 20, 40, 50, 75, 90, 95, or 100% of the mRNA molecules or proteins whose expression or activity is modulated by the therapy are molecules that had altered expression levels compared to the corresponding levels in healthy patients. Preferably, the therapy modulates the expression or activity of at least 20, 40, 50, 75, 90, 95, or 100% of all of the mRNA molecules or proteins that had altered expression levels compared to the corresponding levels in healthy patients. In another preferred embodiment, the therapy modulates the expression or activity of at least 20, 40, 50, 75, 90, 95, or 100% of all of the cancer or angiogenesis related mRNA molecules or proteins that had altered expression levels compared to the corresponding levels in healthy patients.

In particular embodiments, the therapy includes at least 3, 5, 7, 10, 15, 20 or more compounds, such as signal transduction inhibitors. In particular embodiments, the pharmaceutically active compounds in the therapy only include signal transduction inhibitors. In other embodiments, the therapy includes both signal transduction inhibitors and other chemotherapeutic agents, such as cytotoxic agents or immunotherapy agents. Preferred therapies include the signal transduction inhibitor Herceptin or any other compound (e.g., a monoclonal or polyclonal antibody) that binds or inhibits Her2 (U.S. Pat. No. 6,165,464). Another preferred signal transduction inhibitor is ^{Pr}Gleevec™ (imatinib mesylate, Novartis Pharmaceuticals Canada Inc., Dorval, Quebec). Preferred combination therapies include Herceptin and/or ^{Pr}Gleevec™. Other exemplary therapies include an anti-vascular agent, cyclophosphamide, and/or vinblastine. It is also contemplated that the therapy can include an agent (e.g., naked DNA, a DNA vector, or a viral vector) that inactivates a gene that promotes cancer (e.g., an oncogene) or excessive angiogenesis or a gene that is expressed at a higher than normal level in a subject, or the therapy can include an agent (e.g., naked DNA, a DNA vector, or a viral vector encoding an mRNA or protein of interest or containing a promoter that integrates upstream of an endogenous gene of interest) that increases the expression of a gene that inhibits cancer (e.g., a tumor suppressor gene) or excessive angiogenesis or a gene that is expressed at a lower than normal level in a subject.

In preferred embodiments, the therapy is administered using an extended release device. In certain embodiments, the therapy is administered orally, intramuscularly, intravenously, subcutaneously, or by inhalation.

Exemplary cancers that can be treated, stabilized, or prevented using the above methods include prostate cancers, breast cancers, ovarian cancers, pancreatic cancers, gastric cancers, bladder cancers, salivary gland carcinomas, gastrointestinal cancers, lung cancers, colon cancers, melanomas, brain tumors, leukemias, lymphomas, and carcinomas. The cancer may or may not be a hormone related or dependent cancer (e.g., an estrogen or androgen related cancer). Benign tumors may also be treated or prevented using the methods and compositions of the present invention. Preferably, the therapy inhibits angiogenesis of the cancer (e.g., inhibits the rate of blood cell formation or decreases the number or size of blood vessels) by at least 10, 25, 40, 50, 60, 70, 80, 90, 95, or 100%. Other exemplary angiogenesis related diseases that can be treated or prevented using the methods of the invention are listed in FIG. 4. Preferred mammals include humans and mammals of veterinary interest.

As used herein, by “higher than normal expression level” is meant expression of an mRNA or protein at a level that is higher that the average expression level of the corresponding molecule in healthy subjects. In various embodiments, the expression level is at least 20, 40, 50, 75, 90, 100, 200, 500, or even 1000% higher than the level in control subjects.

By “lower than normal expression level” is meant expression of an mRNA or protein at a level that is lower that the average expression level of the corresponding molecule in healthy subjects. In various embodiments, the expression level is at least 20, 40, 50, 75, 90, 95, or 100% lower than the level in control subjects. In some embodiments, the expression of the mRNA or protein is not detectable.

By “modulate expression or activity” is meant to either increase or decrease expression or activity, for example, of a protein or nucleic acid sequence, relative to control conditions. The modulation in expression or activity is preferably an increase or decrease of at least 20, 40, 50, 75, 90, 100, 200, 500, or even 1000%. In various embodiments, transcription, translation, mRNA or protein stability, or the binding of the mRNA or protein to other molecules in vivo is modulated by the therapy. The level of mRNA may be determined by standard Northern blot analysis, and the level of protein may be determined by standard Western blot analysis, such as the analyses described herein or those described by, for example, Ausubel et al. (Current Protocols in Molecular Biology, John Wiley & Sons, New York, 2000). In one embodiment, the level of a protein is determined by measuring the level of enzymatic activity, using standard methods. In another preferred embodiment, the level of mRNA, protein, or enzymatic activity is equal to or less than 20, 10, 5, or 2-fold above the corresponding level in control cells that do not express a functional form of the protein, such as cells homozygous for a nonsense mutation. In yet another preferred embodiment, the level of mRNA, protein, or enzymatic activity is equal to or less than 20, 10, 5, or 2-fold above the corresponding basal level in healthy cells that have not been exposed to conditions that induce abnormal cell proliferation or that inhibit apoptosis.

By a “dosage sufficient to modulate mRNA or protein expression or activity” is meant an amount of a therapy that increases or decreases mRNA or protein expression or activity when administered to a subject. Preferably, for a compound that decreases expression or activity, the modulation is a decrease in expression or activity that is at least 10%, 30%, 40%, 50%, 75%, or 90% lower in a treated subject than in the same subject prior to the administration of the inhibitor or than in an untreated, control subject. In addition, preferably, for a compound that increases expression or activity, the amount of expression or activity of the mRNA or protein is at least 1.5-, 2-, 3-, 5-, 10-, or 20-fold greater in a treated subject than in the same subject prior to the administration of the modulator or than in an untreated, control subject.

By “treating, stabilizing, or preventing cancer” is meant causing a reduction in the size of a tumor, slowing or preventing an increase in the size of a tumor, increasing the disease-free survival time between the disappearance of a tumor and its reappearance, preventing an initial or subsequent occurrence of a tumor, or reducing an adverse symptom associated with a tumor. In one preferred embodiment, the percent of cancerous cells surviving the treatment is at least 20, 40, 60, 80, or 100% lower than the initial number of cancerous cells, as measured using any standard assay. Preferably, the decrease in the number of cancerous cells induced by administration of a therapy of the invention is at least 2, 5, 10, 20, or 50-fold greater than the decrease in the number of non-cancerous cells. In yet another preferred embodiment, the number of cancerous cells present after administration of a therapy is at least 2, 5, 10, 20, or 50-fold lower than the number of cancerous cells present after administration of a vehicle control. Preferably, the methods of the present invention result in a decrease of 20, 40, 60, 80, or 100% in the size of a tumor as determined using standard methods. Preferably, at least 20, 40, 60, 80, 90, or 95% of the treated subjects have a complete remission in which all evidence of the cancer disappears. Preferably, the cancer does not reappear or reappears after at least 5, 10, 15, or 20 years. In another preferred embodiment, the length of time a patient survives after being diagnosed with cancer and treated with a therapy of the invention is at least 20, 40, 60, 80, 100, 200, or even 500% greater than (i) the average amount of time an untreated patient survives or (ii) the average amount of time a patient treated with another therapy survives.

By an “estrogen-related cancer” is meant a cancer that is modulated by estrogen. Examples of estrogen-related cancers include, without limitation, breast cancer and ovarian cancer. Her2 is overexpressed in many estrogen-related cancers (U.S. Pat. No. 6,165,464).

By an “androgen-related cancer” is meant a cancer that is modulated by androgen. An example of androgen-related cancers is prostate cancer.

By “cancer related gene” is meant a gene associated with an altered risk for a cancer or an altered prognosis for a cancer. Exemplary cancer related genes that promote cancer include oncogenes; genes that enhance cell proliferation, invasion, or metastasis; genes that inhibit apoptosis; and pro-angiogenesis genes. Cancer related genes that inhibit cancer include, but are not limited to, tumor suppressor genes; genes that inhibit cell proliferation, invasion, or metastasis; genes that promote apoptosis; and anti-angiogenesis genes.

By “angiogenesis” is meant the formation of new blood vessels and/or the increase in the volume, diameter, length, or permeability of existing blood vessels, such as blood vessels in a tumor or between a tumor and surrounding tissue. Angiogenesis is associated with a variety of neoplastic and non-neoplastic disorders.

By “an angiogenesis related disease” is meant a disease associated with excessive or insufficient blood vessel growth, an abnormal blood vessel network, and/or abnormal blood vessel remodeling. For example, insufficient vascular growth can lead to decreased levels of oxygen and nutrients, which are required for cell survival. Angiogenesis also contributes to tumor growth. Other exemplary angiogenesis related diseases are listed in FIG. 4.

By “treating, stabilizing, or preventing an angiogenesis related disease” is meant modulating the formation of new blood vessels and/or modulating the volume, diameter, length, permeability, or number of existing blood vessels. In preferred embodiments, an initial or subsequent occurrence of an angiogenesis related disorder is prevented or an adverse symptom associated with an angiogenesis related disorder is reduced. Preferably, the methods of the present invention result in a modulation of 20, 40, 60, 80, 100, 500, or even 1000% in the volume, diameter, length, permeability, and/or number of blood vessels as determined using standard methods. Preferably, at least 20, 40, 60, 80, 90, or 95% of the treated subjects have a complete remission in which all evidence of the disease disappears. In another preferred embodiment, the length of time a patient survives after being diagnosed with an angiogenesis related disease and treated with a therapy of the invention is at least 20, 40, 60, 80, 100, 200, or even 500% greater than (i) the average amount of time an untreated patient survives or (ii) the average amount of time a patient treated with another therapy survives.

By “combination therapy” is meant a combination of two or more compounds that each modulate the expression or activity of an mRNA or protein (e.g., cancer or angiogenesis related molecules). The compounds may directly or indirectly modulate the expression or activity of the mRNA or protein. For example, a compound may indirectly modulate the expression or activity of an mRNA or protein of interest by modulating the expression or activity of a molecule (e.g., a nucleic acid, protein, signaling molecule, growth factor, cytokine, or chemokine) that directly or indirectly affects the expression or activity of the mRNA or protein of interest. In some embodiments, the compounds inhibit cell division or induce apoptosis. These compounds in the therapy may include, for example, unpurified or purified proteins, antibodies, synthetic organic molecules, naturally-occurring organic molecules, nucleic acid molecules, and components thereof. The compounds in a combination therapy may be administered simultaneously or sequentially. Preferred compounds are signal transduction inhibitors.

By “signal transduction inhibitor” is meant a compound that inhibits a pathway involved in modulating intracellular events in response to an extracellular signal, such as the binding of a hormone to a cell-surface receptor. In some cases, the binding of a ligand to a cell-surface receptor modulates the phosphorylation state of the intracellular domain of the receptor or the phosphorylation state of proteins that interact with the intracellular domain of the receptor. The change in phosphorylation state of the protein or other downstream proteins often results in either the activation of a transcription factor and an increase in gene expression or the inhibition of a transcription factor and a decrease in gene expression. Thus, a signal transduction inhibitor typically effects a change in the expression of one or more genes. For example, signal transduction inhibitors may inhibit an activity of one or more molecules in a signal transduction pathway (e.g., an extracellular ligand such as a hormone, a cell-surface receptor, a kinase, a phosphatase, or a transcription factor). In contrast, many cytotoxic agents nonspecifically kill proliferating cells and may nonspecifically inhibit the expression of a variety of genes.

By “purified” is meant separated from other components that naturally accompany it. Typically, a factor is substantially pure when it is at least 50%, by weight, free from proteins, antibodies, and naturally-occurring organic molecules with which it is naturally associated. Preferably, the factor is at least 75%, more preferably, at least 90%, and most preferably, at least 99%, by weight, pure. A substantially pure factor may be obtained by chemical synthesis, separation of the factor from natural sources, or production of the factor in a recombinant host cell that does not naturally produce the factor. Proteins and small molecules may be purified by one skilled in the art using standard techniques such as those described by Ausubel et al. (Current Protocols in Molecular Biology, John Wiley & Sons, New York, 2000). The factor is preferably at least 2, 5, or 10 times as pure as the starting material, as measured using polyacrylamide gel electrophoresis, column chromatography, optical density, HPLC analysis, or western analysis (Ausubel et al., supra). Preferred methods of purification include immunoprecipitation, column chromatography such as immunoaffinity chromatography, magnetic bead immunoaffinity purification, and panning with a plate-bound antibody.

By “mutation” is meant an alteration in a naturally-occurring or reference nucleic acid sequence, such as an insertion, deletion, frameshift mutation, silent mutation, nonsense mutation, or missense mutation. Preferably, the amino acid sequence encoded by the nucleic acid sequence has at least one amino acid alteration from a naturally-occurring sequence.

The present invention provides a number of advantages related to the treatment and prevention of cancers and other angiogenesis related diseases. For example, the methods can be generally applied to the treatment of malignant or benign tumors of any cell, tissue, or organ type. The simultaneous or sequential use of multiple therapeutic agents (e.g., signal transduction inhibitors) should greatly reduce the incidence of cancer and reduce the number of treated cancers that become resistant to therapy. In addition, therapeutic agents that are used as part of a combination therapy may require a lower dose to treat a cancer or angiogenesis related disease than the corresponding dose required when the therapeutic agents are used individually. The low dose of each compound in the combination therapy reduces the severity of potential adverse side-effects from the compounds. Clinical trials involving combination therapies are also less expensive than performing multiple clinical trials that each involve only one chemotherapeutic agent.

Other features and advantages of the invention will be apparent from the following detailed description and from the claims.

### BRIEF DESCRIPTION OF THE DRAWINGS

FIGS. 1A-1J illustrate the effects of Herceptin on tumor growth, angiogenesis, and gene expression. FIG. 1A is a set of pictures of the architecture of the MDA-MB-361HK tumor vasculature on treatment day 15 visualized by fluorescence microscopy. The scale bar indicates 100 μm. FIG. 1B is a graph illustrating that the mean vessel diameter was significantly reduced in the Herceptin-treated group (open circle, n=6) compared to the control antibody-treated group (filled circle, n=6). FIG. 1C is a bar graph showing that vascular permeability was significantly reduced in the Herceptin-treated group (open column, n=6) compared to the control antibody-treated group (filled column, n=6) at day 15. FIG. 1D is a graph illustrating that tumor size was significantly reduced in the Herceptin-treated group (open circle, n=6) compared to the control antibody-treated group (filled circle, n=6). FIG. 1E is a graph showing that survival was significantly increased in the Herceptin-treated group (open column, n=6) compared to the control antibody-treated group (filled column, n=6). FIG. 1F is a set of bar graphs showing that when the tumor reached 6 mm size, mean vessel diameter, vessel volume, and vascular permeability were significantly reduced in the Herceptin treated group (open column, n=6) compared to the control antibody treated group (filled column, n=6), while vessel length was not significantly different. In FIG. 1B-1F, the data are shown as the mean±standard deviation, * P<0.05, Mann-Whitney U test (StatView, Abacus, Berkeley, Calif.), compared with the corresponding values in the Herceptin-treated group. In FIGS. 1B and 1D, data up to treatment day 15 are plotted since the first animal was sacrificed on treatment day 15. FIG. 1G is a set of pictures of the expression profiling of 23 angiogenesis related genes performed using the Human Cancer/angiogenesis-2 GEArray kit (SuperArray Inc., Bethesda, Md.) according to manufacturer's instructions. Expression of vascular endothelial growth factor (VEGF), transforming growth factor alpha (TGF-α), angiopoietin-1 (Ang-1), and plasminogen activator inhibitor-1 (PAI-1) decreased, and expression of thrombospondin-1 (TSP-1) increased in the Herceptin treated tumors compared to the control tumors in vivo (left 2 columns). The expression profiles were confirmed by Northern blot analysis in vivo and in vitro (right 2 columns). “C” denotes the control antibody-treated group, and “H” denotes the Herceptin-treated group. Quantitative analysis was performed by densitometry. The abundance of mRNA was normalized to that of β-Actin. FIG. 1H is a picture of the detection of HER2 expression using the A0485 antibody (Dako). HER2 was observed in the majority of MDA-MB-361-HK cells grown in the SCID mouse cranial window, primarily in the cell membrane in both groups. FIG. 1I is a picture of VEGF expression that was detected using the 3E7 antibody (gift from Drs. Rolf Brekken and Philip Thorpe). VEGF was observed in the host stromal cells (arrows) as well as in the tumor cells in both groups. The images are from Herceptin treated tumors. The scale bar indicates 50 μm. FIG. 1J is a table illustrating the effect of Herceptin on tumor blood vessels and expression of angiogenic factors.

FIGS. 2A-2G are tables listing various angiogenesis inhibitors that may be used in the therapies of the invention (Carmeliet and Jain, Nature 407:249-257, 2000).

FIG. 3 is a table listing many proteins that activate or inhibit angiogenesis. The expression or activity of these factors can be modulated using the methods of the invention (Carmeliet and Jain, Nature 407:249-257, 2000).

FIG. 4 is a table listing many angiogenesis related diseases that can be treated or prevented using the methods and compositions of the invention (Carmeliet and Jain, Nature 407:249-257, 2000).

### DETAILED DESCRIPTION

The present invention is based, in part, on the discovery that the signal transduction inhibitor Herceptin modulates the expression of multiple cancer and angiogenesis related genes. Thus, Herceptin offers an attractive alternative to using multiple therapeutic agents to modulate expression of these genes. Because Herceptin is a monoclonal antibody against human epidermal growth factor receptor 2 (HER2) which is expressed in a variety of tumors, Herceptin may be generally used to treat numerous cancers and angiogenesis related diseases (Slamon et al., N. Engl. J. Med. 344:783-792, 2001).

Based on this surprising discovery that Herceptin modulates the expression of multiple pro- and anti-angiogenic factors, we expect other compounds (e.g., other signal transduction inhibitors) to also modulate the expression of multiple cancer and angiogenesis related genes. Thus, combination therapies that include two or more such compounds should have increased drug efficacy because of the ability to modulate the expression or activity of numerous cancer and angiogenesis related genes. Preferred signal transduction inhibitors or preferred combination therapies (e.g., combinations of signal transduction inhibitors) can be selected for a particular patient by determining the expression profile of cancer or angiogenesis related genes in the patient and selecting a therapy that (i) inhibits the expression of genes that promote cancer or angiogenesis and that are expressed at a high level in the patient and/or (ii) promotes the expression of genes that inhibit cancer or angiogenesis and that are expressed at a low level in the patient. Preferably, the signal transduction inhibitor or combination therapy normalizes the expression or activity of the majority or all of the cancer or angiogenesis related genes that are expressed at an altered level in the patient and has negligible effect on other genes.

These methods are described further below.

Inhibition of Angiogenesis by Herceptin

The ability of Herceptin treatment to induce normalization and regression of the vasculature of a HER2 over-expressing human breast carcinoma was tested in vivo. For better in vivo growth of tumors for this analysis, the MDA-MB-361HK cell line (Genentech Inc., San Francisco, Calif.) was derived from MDA-MB-361, a HER2 over-expressing human breast cancer cell line derived from a brain metastasis. MDA-MB-361HK tumor xenografts were established by injection of a single-cell suspension into the dorsal flank of female SCID mice supplemented with an estrogen pellet (17-β-estradiol 0.36 mg, Innovative Research, Sarasota, Fla.) inserted subcutaneously a few days before cell injection. Tumors were serially transplanted up to four generations. Cranial windows were prepared in 8-10 week old female SCID mice as previously described (Jain et al., in Tumor Models In Cancer Research, ed. Teicher, Humana Press Inc., Totowa, N.J., pp. 647-671, 2001). These mice were bred and maintained in our gnotobiotic animal facility. A few days after estrogen supplementation, a piece of MDA-MB-361HK tumor, approximately 1 mm in diameter, was implanted into the cranial windows. Approximately 14 days after tumor implantation, when the tumor became well vascularized (approximately 2 mm in diameter; referred to as treatment day 0), we began treating the animals with intraperitoneal injection of either Herceptin or control human IgG (Genentech), 30 mg/kg, every 3 days. Treatment continued until the tumor diameter reached approximately 6 mm, the size of the window. Then, the mouse was sacrificed, and the tumor was resected for histological and molecular analyses. Animal survival was derived from the time interval between the initiation of treatment and animal sacrifice. Tumor size, vessel density, mean vessel diameter, and vessel volume of the cranial window tumor were analyzed every 3 days from treatment day 0 using intravital fluorescence microscopy and quantified as previously described (Jain et al., supra). For visualization of blood vessels, 10 mg/ml fluorescein isothiocyanate labelled dextran solution (Mᵣ, 2,000,000; Sigma Chemical Co., St. Louis, Mo.) was injected via a tail vein cannula. Vascular permeability to albumin was measured on treatment day 0, day 15, and the day the tumor reached 6 mm, as previously described (Jain et al., supra). Mice were injected with a bolus (100 μl) of 1% tetramethylrhodamine labelled bovine serum albumin (Molecular Probes, Eugene, Oreg.) in saline via the tail vein.

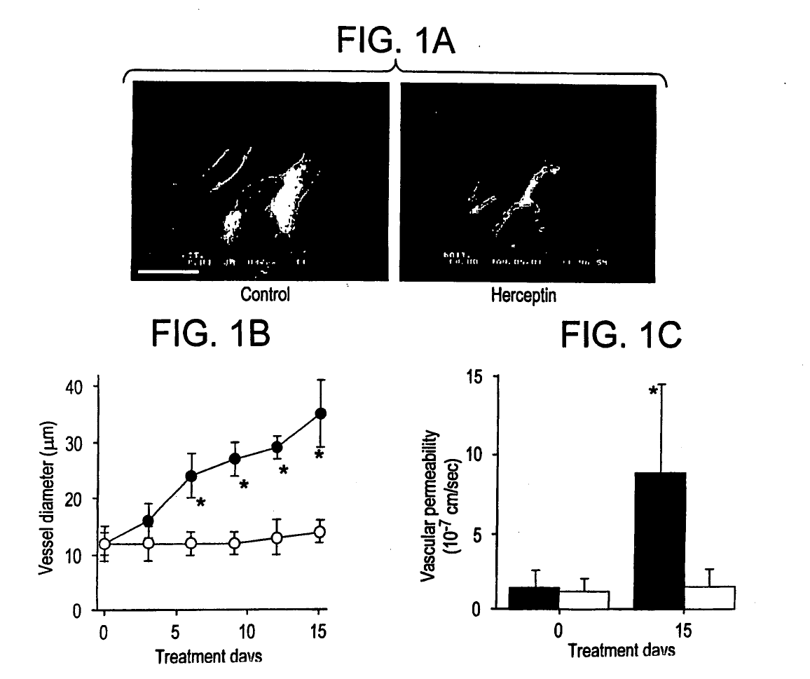

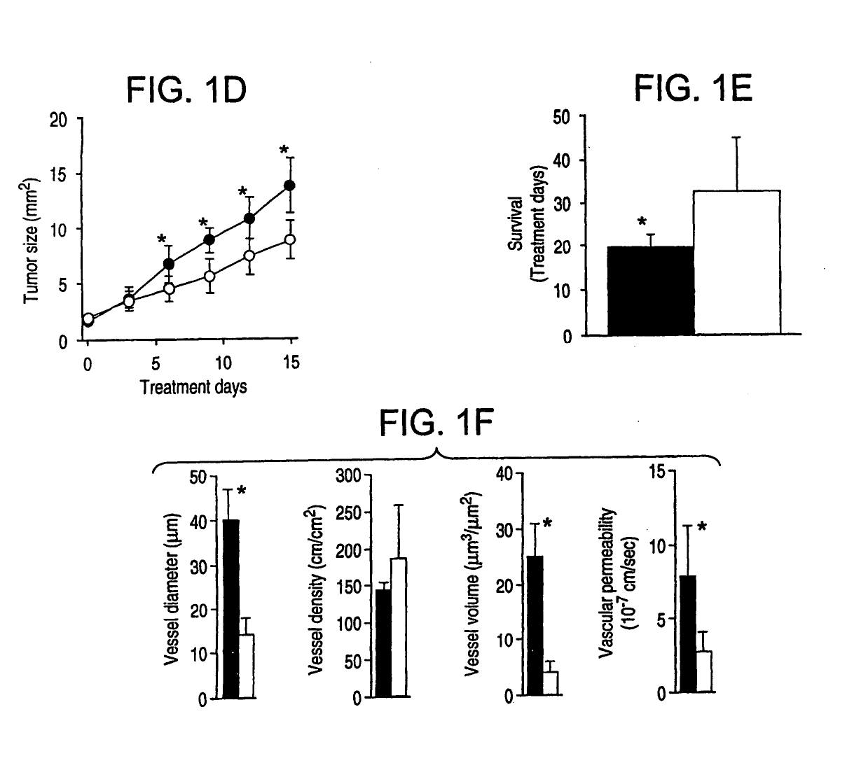

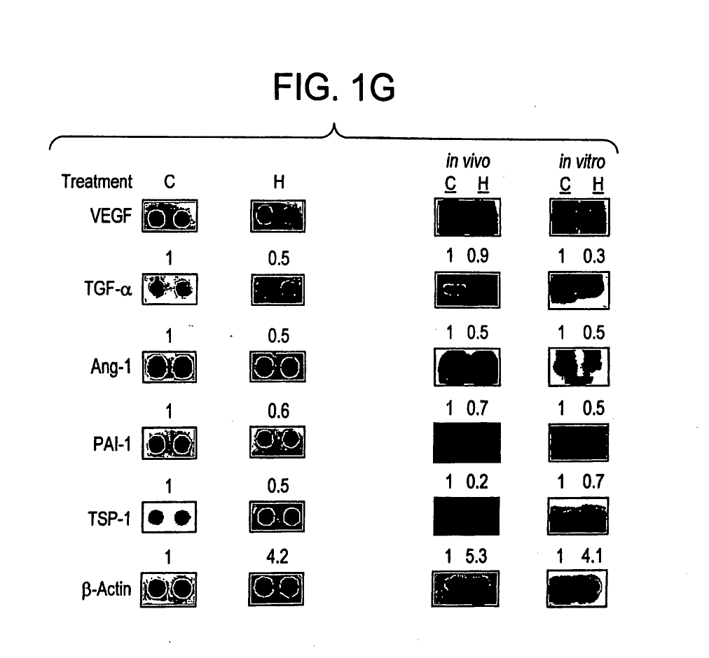

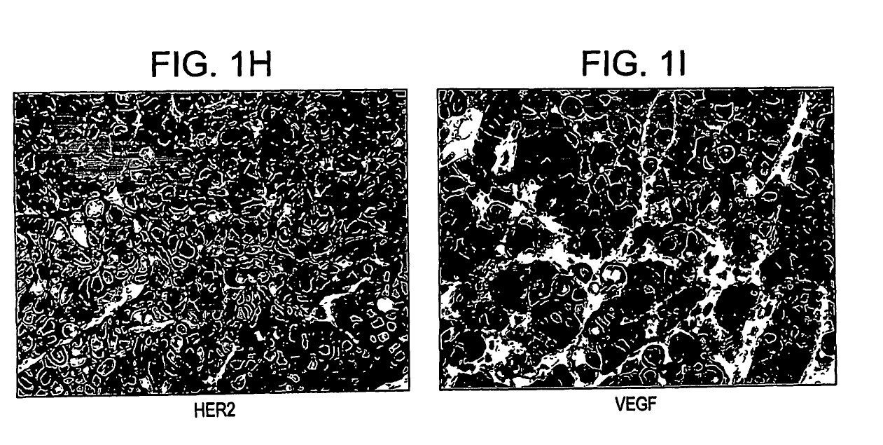

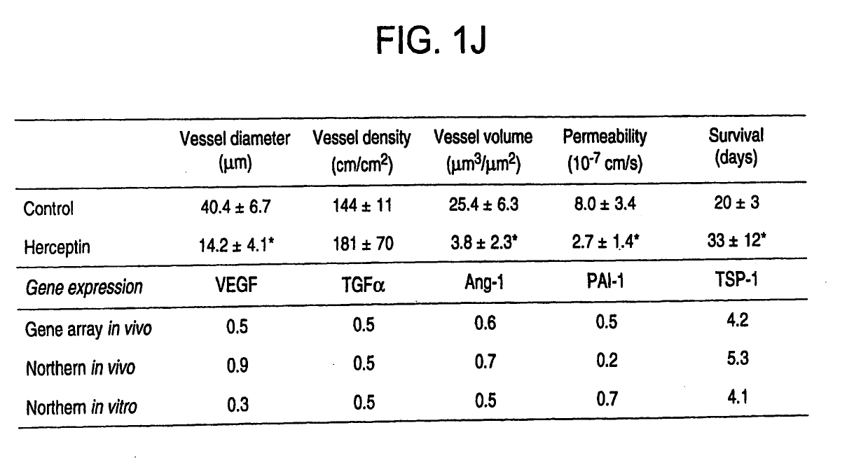

Based on the above analysis, Herceptin treatment significantly reduced the diameter and volume of tumor vessels compared to control antibody treatment, while not significantly affecting the length of the vessels (FIGS. 1A and 1B). Vascular permeability was also significantly reduced by Herceptin treatment (FIGS. 1C and 1J; 8.8±5.7 and 1.6±1.1×10⁻⁷ cm s⁻¹ at day 15 in the control and herceptin groups, respectively). Thus, the vessels in the Herceptin treated tumors more closely resembled a normal phenotype. Tumor growth was delayed and animal survival was significantly extended (FIGS. 1D, 1E, and 1J). These vascular effects were not tumor-size dependent as evidenced by the sustained differences at maximum (6 mm) tumor size (FIG. 1F). The more efficient blood vessel network resulting from Herceptin treatment more closely resembles normal networks, and thus, may improve drug delivery to previously inaccessible regions.

Modulation of Multiple Pro- and Anti-Angiogenic Factors by Herceptin

Based on the finding that the vascular effect of Herceptin was very stable throughout the course of the study, together with the known diversity of human epidermal growth factor receptor pathways (Yarden et al., Nat. Rev. Mol. Cell Biol. 2:127-137, 2001), we hypothesized that Herceptin treatment affects multiple pro- and anti-angiogenic factors. To test this hypothesis, we examined 23 angiogenesis related genes using the Human Cancer/angiogenesis-2 GEArray kit (SuperArray Inc., Bethesda, Md.) according to manufacturer's instructions. For this analysis, the gene expression of tumors in vivo and in vitro was measured in the presence and absence of Herceptin. In particular, tumors were removed from Herceptin and control antibody treated mice once the tumors reached 6 mm in size. The tumor tissue was then homogenized, and the cells were lysed. To collect mRNA, the lysate was applied to a spin column containing an oligo-T matrix to bind the polyA termini of the mRNA. The mRNA was then eluted from the matrix, and the mRNA was reversed transcribed in the presence of [³²P]-dCTP to generate radiolabelled cDNA. A 1 μg sample of cDNA was hybridized to the chip overnight, and then the radioactive cDNA bound to the chip was detected. To study the effect of Herceptin on tumor cells in vitro, MDA-MB-361HK cells were plated at a density of 2×10⁶ cells/10 cm dish overnight. The cells were treated with 50 μg/ml Herceptin or control human IgG for 72 hours, and the cell lysate was collected for Northern blot analysis and for DNA chip analysis, as described above.

This expression profiling demonstrated that VEGF, transforming growth factor alpha (TGF-α), angiopoietin-1 (Ang-1), and plasminogen activator inhibitor-1 (PAI-1) was decreased in Herceptin treated tumors. In contrast, expression of thrombospondin-1 (TSP-1) increased in Herceptin treated tumors compared to control tumors in vivo (FIGS. 1G and 1J). These results were subsequently confirmed by Northern blot analysis (FIG. 1G).

HER2 signaling is known to control VEGF and PAI-1 expression. HER2 may affect TGF-α level through interactions with HER1, and may possibly mediate TSP-1 expression through pathways similar to Ras. To our knowledge, no correlation has been reported between HER2 and Ang-1. The vascular effects of Herceptin in the present study are consistent with the reports that Ang-1 increases vessel diameter (Thurston et al., Science 286:2511-2514, 1999), whereas TSP-1 decreases vessel diameter (Bornstein, J. Clin. Invest. 107:929-934, 2001). Vascular permeability is possibly affected by TSP-1 through recruitment of perivascular cells, balanced by the decrease in Ang-1. In this study, Northern blot analysis indicated that VEGF expression was decreased by Herceptin treatment in vitro but not in vivo, suggesting VEGF compensation from host cells. Indeed, VEGF expression was observed by immunohistochemistry in host stromal cells as well as in the tumor cells (FIGS. 1H and 1I). Also, cultured tumor cells expressed lower levels of Ang-1 and PAI-1 and expressed a higher level of TSP-1 in vitro compared to in vivo tumor tissues. The Herceptin mediated change in PAI-1 expression was greater in vivo compared to in vitro. We speculate that Herceptin treatment may have affected PAI-1 from the host through modulation of tumor derived TGF-α. These results collectively indicate significant contribution of tumor-host interaction in the expression of pro- and anti-angiogenic factors.

Selection of Therapies

Instead of, or in addition to, Herceptin, other signal transduction inhibitors can be used for the treatment or prevention of cancers and angiogenesis related diseases. For example, additional therapeutic agents can be administered to inhibit other angiogenic factors, such as angiogenic factors from host cells. Thus, individual or multiple signal transduction inhibitors can be used to achieve stable, long-term vessel normalization and regression.

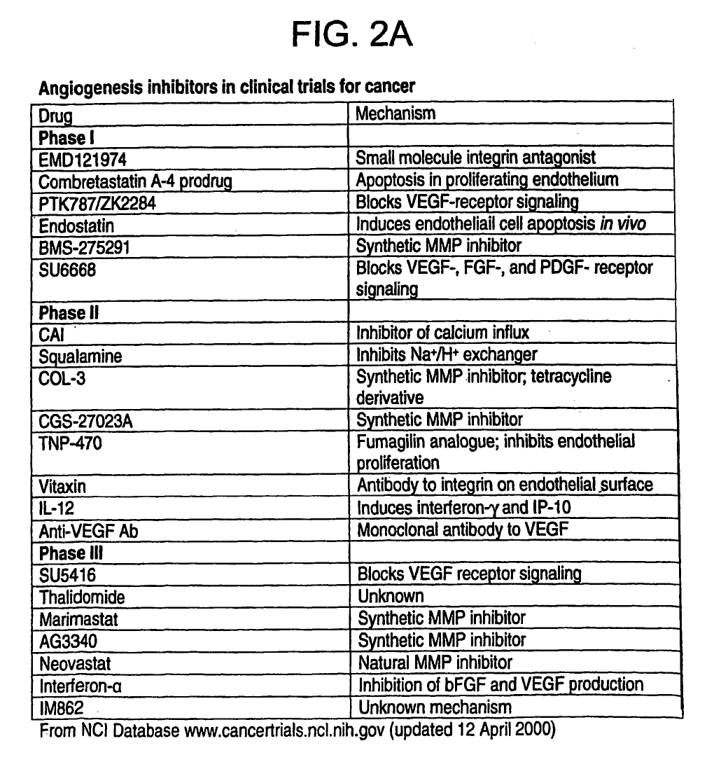

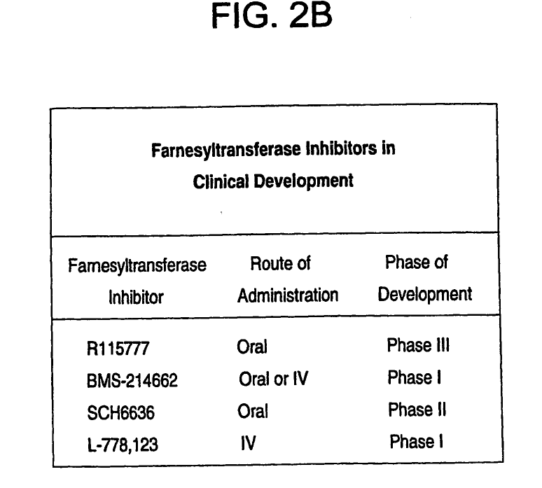

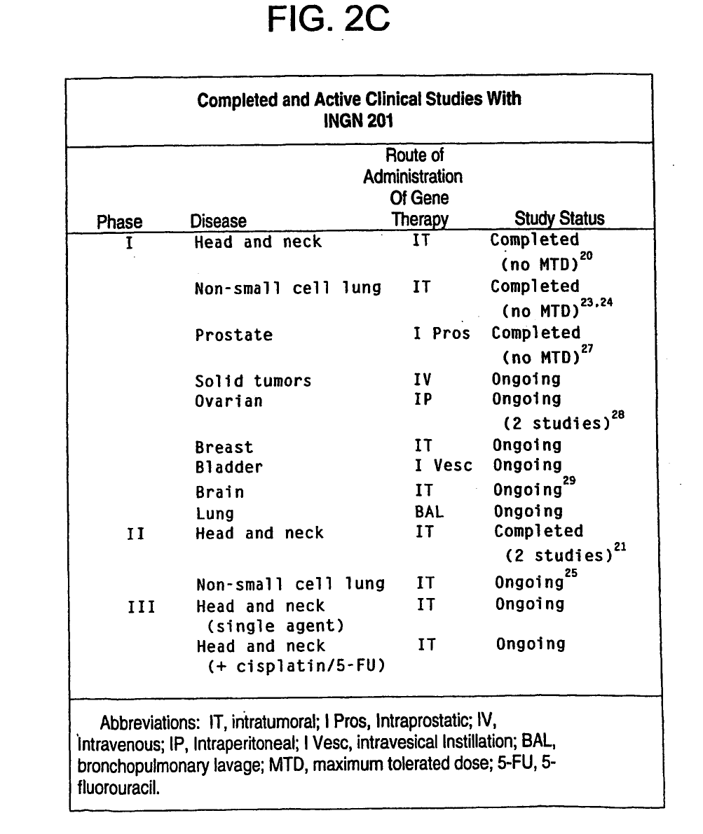

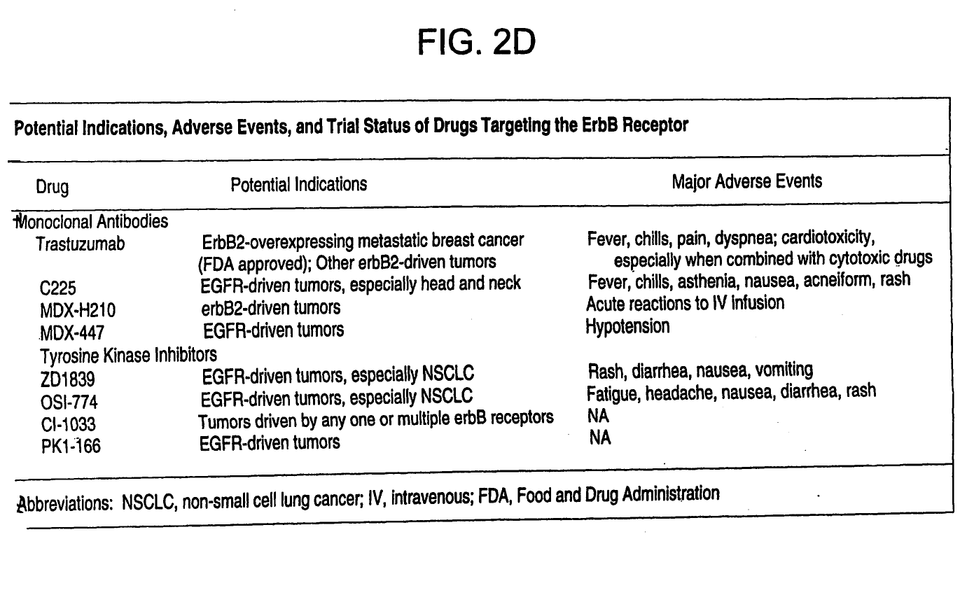

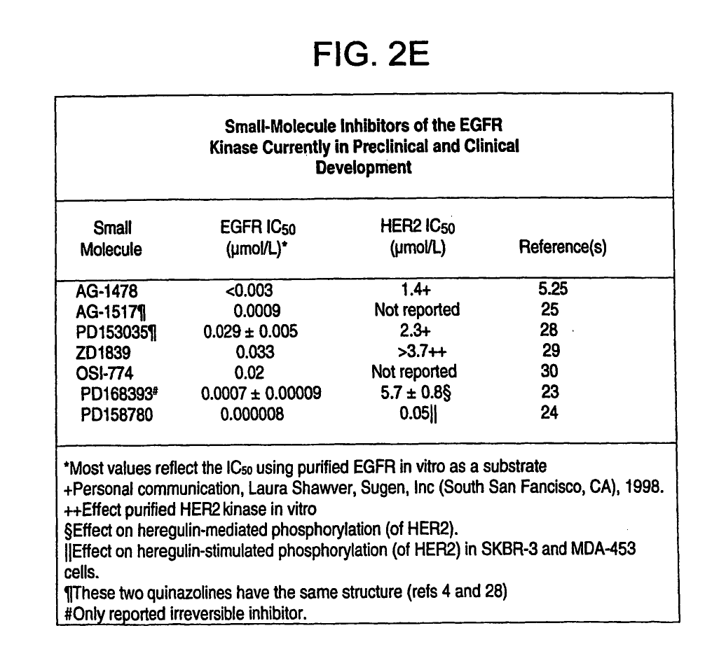

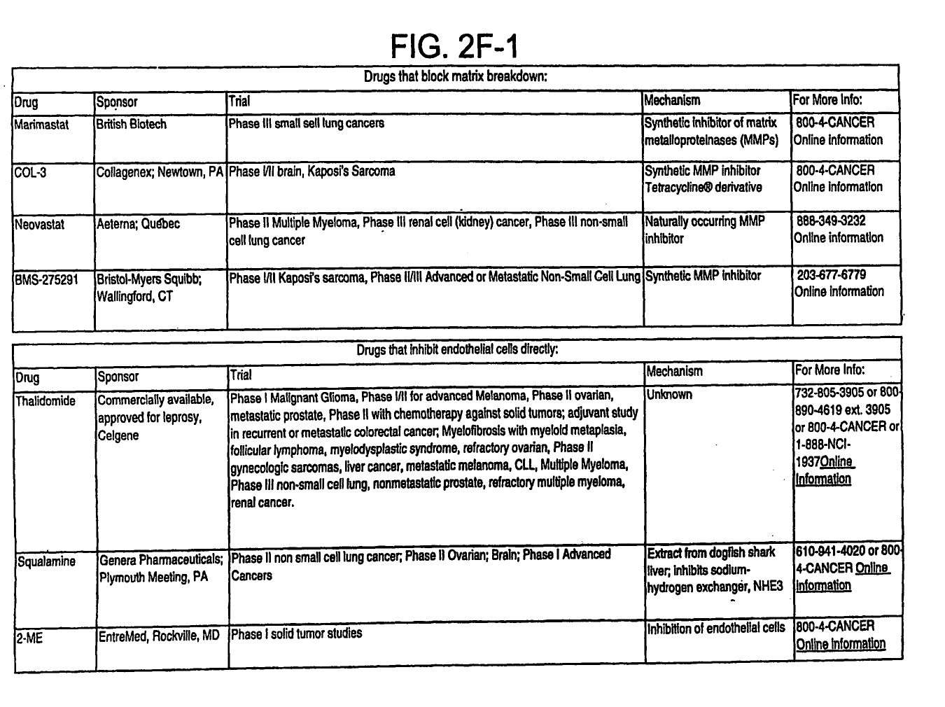

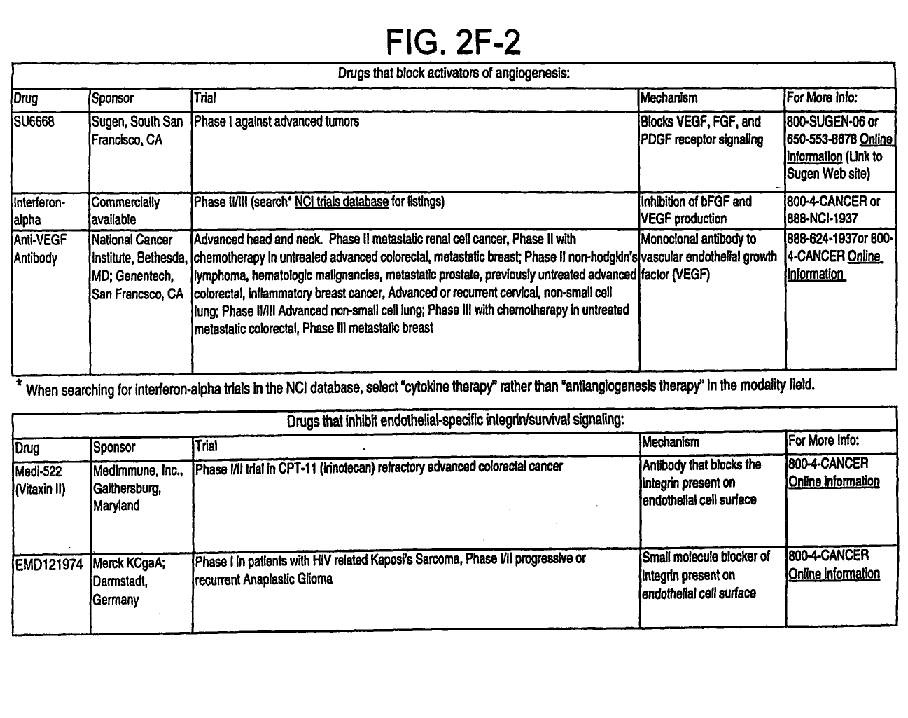

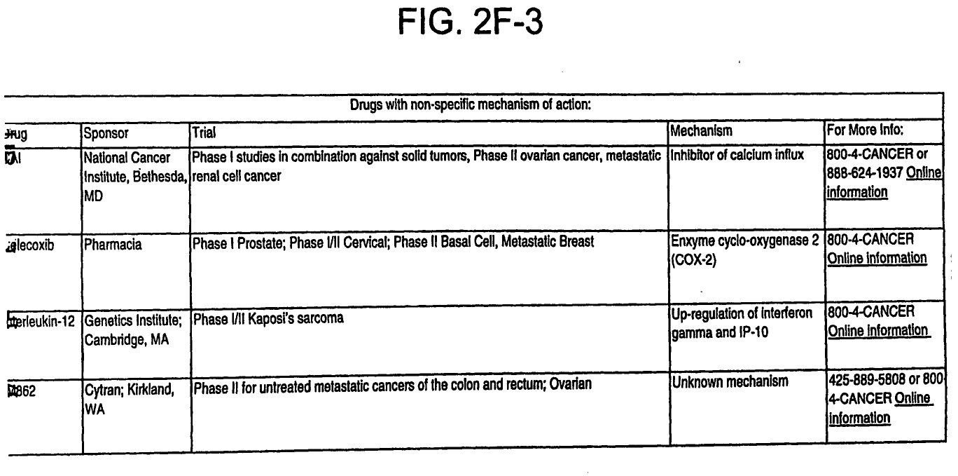

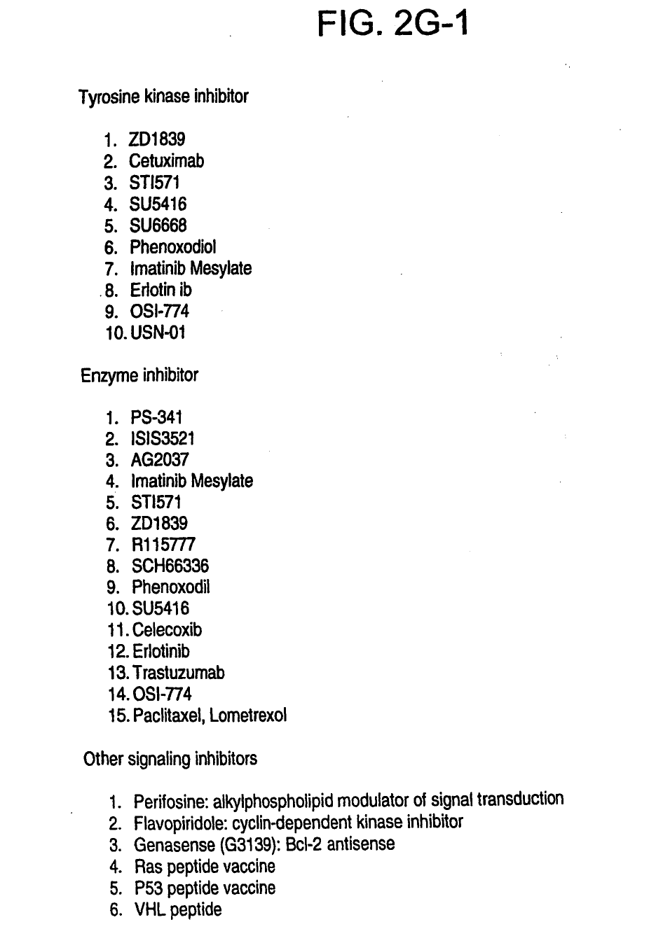

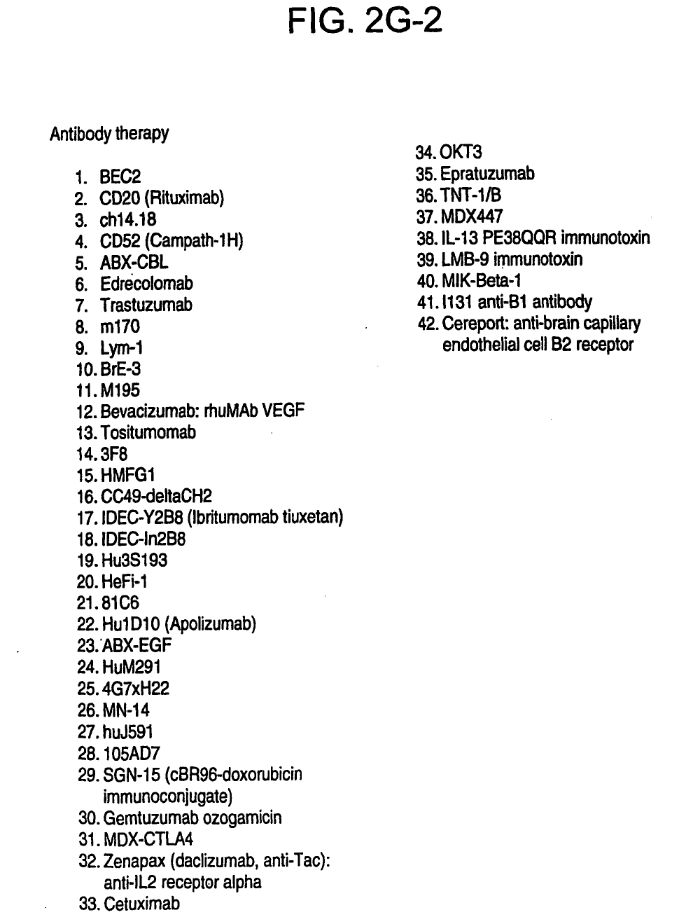

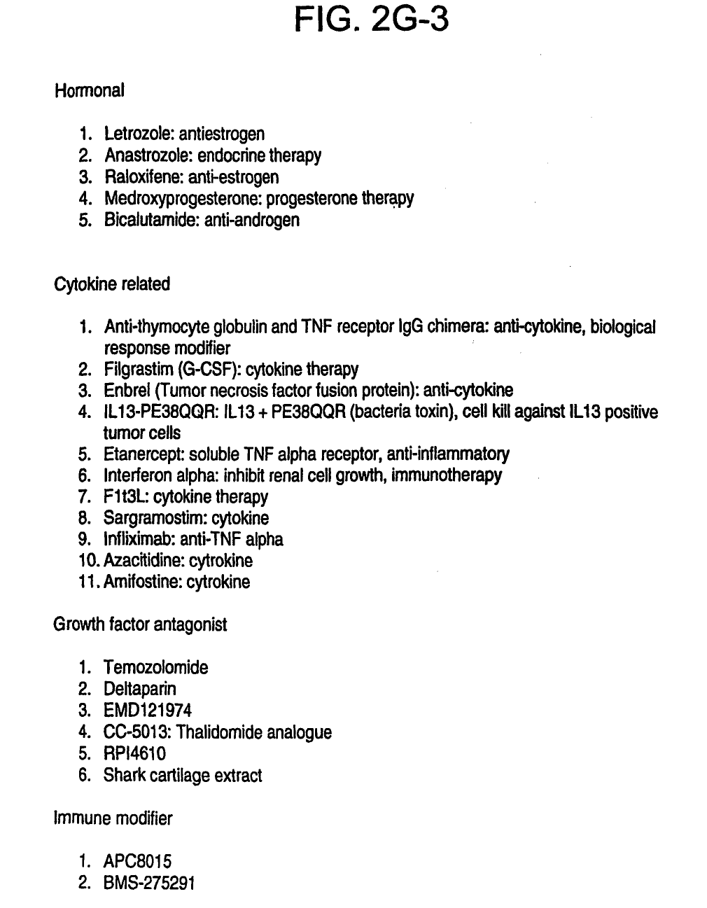

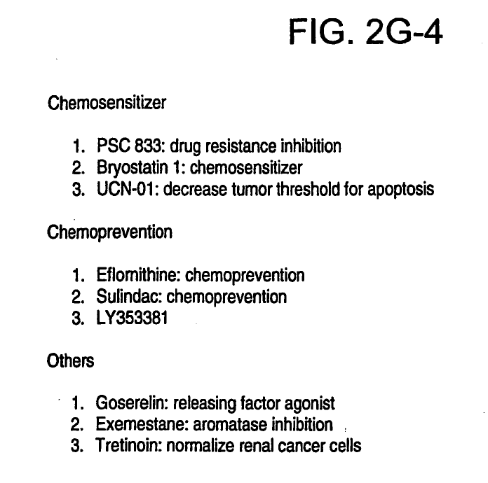

For the selection of a preferred signal transduction inhibitor or a preferred combination therapy for a particular patient, the effect of other compounds (e.g., known signal transduction inhibitors such as those listed in FIGS. 2A-2G or compounds other than signal transduction inhibitors) on gene expression may be determined. For example, the ability of other compounds to modulate the expression of angiogenesis related genes or other cancer related genes may be determined using a DNA chip, as described above for Herceptin. In one exemplary approach, a tumor cultured in vitro or a tumor in an animal model (e.g., a SCID mouse) can be contacted with the compound. Exemplary doses for the administration of an anti-cancer or angiogenesis compound to a SCID mouse include the following: 50 mg/kg (morning) and 100 mg/kg (evening) p.o. for ^{Pr}Gleevec™, 1 mg/mouse i.p. every 2 days for C225, and 20 mg/kg/day p.o for ZD 1839. Then, cells from the tumor are lysed, and mRNA is collected from the lysate and reverse transcribed to generate radiolabelled cDNA. The cDNA is applied to the Human Cancer/angiogenesis-2 GEArray kit or to any other DNA chip. For example, cDNA arrays that contain other known cancer or angiogenesis related molecules (e.g., oncogenes or tumor suppressors) or that contain a portion or all of the cDNA molecules expressed by a particular mammal (e.g. a human) can be prepared using standard methods (Marrack et al., Current Opinion in Immunology 12, 206-209, 2000; Harkin, Oncologist. 5:501-507, 2000; Pelizzari et al., Nucleic Acids Res. 28(22):4577-4581, 2000). Because DNA chips are commercially available for a variety of cell types, the radiolabelled cDNA can also be applied to a DNA chip that contains cDNA molecules from the same cell or tissue type as the tumor. The hybridization of the tumor-derived cDNA molecules to the DNA chip is detected using standard methods. Alternatively, the ability of a compound to modulate the expression of mRNA molecules or proteins can be detected using standard Northern or Western analysis.

The lists of mRNA molecules or proteins that are expressed at altered levels in the presence of various compounds can be stored in a database. This database can be used to select individual or combination therapies for the treatment of a particular patient. For example, the expression profiling analysis described above is performed on cells from a patient who may be at risk for cancer or an angiogenesis related disease or performed on cancerous or diseased cells from a patient with cancer or an angiogenesis related disease. The expression of cancer or angiogenesis related genes in the patient sample is compared to the corresponding expression level in a control sample. If desired, the patient sample may also be analyzed for the presence of mRNA or protein molecules that contain a mutation associated with cancer or altered angiogenesis (i) using DNA chip or Northern analysis with hybridization probes specific for the mutant or wild-type forms or (ii) using an antibody specific for the mutant or wild-type forms.

Next, one or more compounds are selected from the database that (i) inhibit the expression or activity of mRNA molecules or proteins that promote cancer or undesired angiogenesis that are expressed at a higher than normal level in the patient sample or (ii) promote the expression or activity of mRNA molecules or proteins that inhibit cancer or undesired angiogenesis that are expressed at a lower than normal level in the patient sample. Additionally, compounds may be selected that inhibit mRNA molecules or proteins that have a mutation that promotes cancer or an angiogenesis related disease and that have an altered or normal level of expression. The database can be used to select the individual or combination therapy that (i) modulates the greatest number of mRNA molecules or proteins that have altered expression levels in the patient or that have mutations associated with cancer or altered angiogenesis and/or (ii) modulates the least number of mRNA molecules or proteins that have normal expression levels in the patient. The selected individual or combination therapy should have high drug efficacy and produce few, if any, adverse side-effects.

As an alternative to the patient-specific analysis described above, DNA chips can be used to compare the expression of mRNA molecules in a particular type of early or late-stage tumor (e.g., breast cancer cells) or angiogenesis related disease tissue to the expression in normal tissue. Based on this analysis, an individual or combination therapy for patients with this type of tumor or disease can be selected to modulate the expression of the mRNA or proteins that have altered expression in this tumor or diseased tissue.

In addition to being used to select a therapy for a particular patient or group of patients, expression profiling can be used to monitor the changes in mRNA and/or protein expression that occur during treatment. For example, expression profiling can be used to determine whether the expression of cancer or angiogenesis related genes has returned to normal levels. If not, the dose of one or more compounds in the therapy can be altered to either increase or decrease the effect of the therapy on the expression levels of the corresponding cancer or angiogenesis related gene(s). In addition, this analysis can be used to determine whether a therapy affects the expression of other genes (e.g., genes that are associated with adverse side-effects). If desired, the dose or composition of the therapy can be altered to prevent or reduce undesired side-effects.

Exemplary Angiogenesis Related Molecules that can be Modulated Using the Methods of the Invention

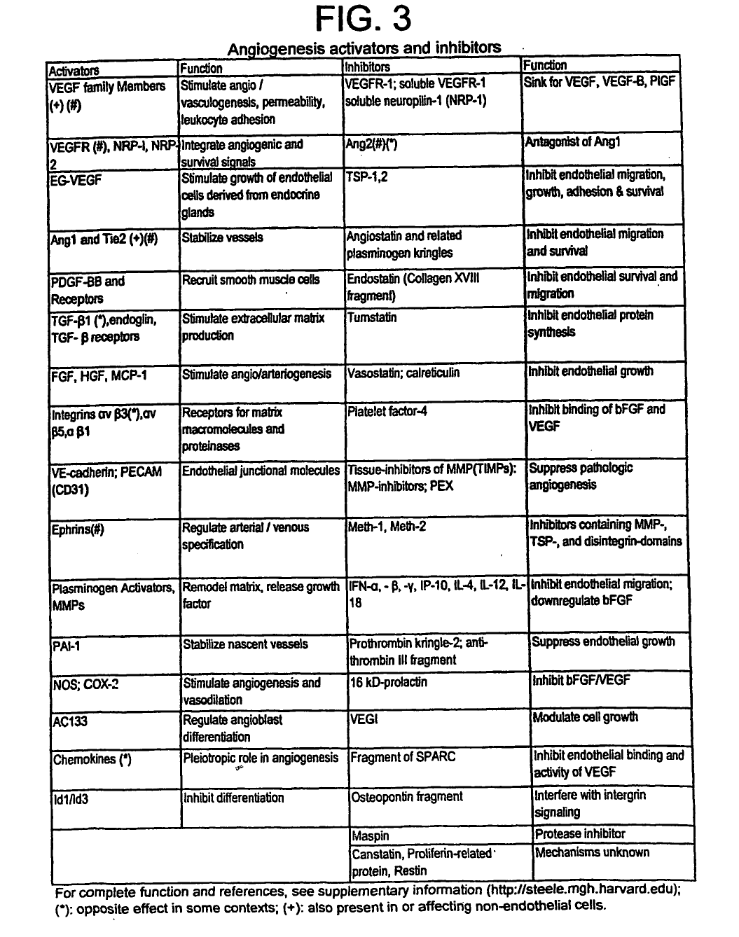

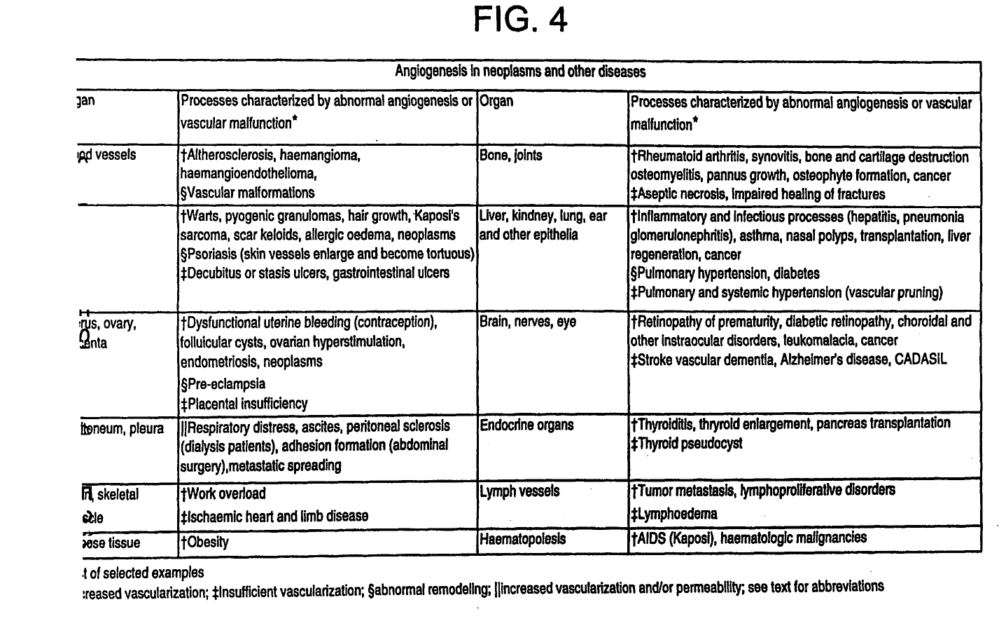

In addition to the angiogenesis related molecules mentioned above, there are numerous other factors that promote or inhibit angiogenesis and that can be modulated using the therapies of the invention (FIG. 3). For example, many growth factors and cytokines exert chemotactic, mitogenic, modulatory, or inhibitory activities on endothelial cells, smooth muscle cell, and fibroblasts and, therefore, can be expected to participate in angiogenic processes. The process involves the concerted action of proteolytic enzymes, extracellular matrix components, cell adhesion molecules, and vasoactive factors. Factors modulating growth, chemotactic behavior, and/or functional activities of smooth muscle cells include Activin A, Adrenomedullin, aFGF, ANF, Angiogenin, Angiotensin-2, Betacellulin, bFGF, CLAF, ECDGF (endothelial cell-derived growth factor), ET (Endothelins), Factor X, Factor Xa, HB-EGF, Heart derived inhibitor of vascular cell proliferation, IFN-gamma, IL1, LDGF (Leiomyoma-derived growth factor), MCP-1, MDGF (macrophage-derived growth factor, monocyte-derived growth factor), nitric oxide (NO), Oncostatin M, PD-ECGF, PDGF, Prolactin, prostacyclin, Protein S, SDGF (smooth muscle cell-derived growth factor), SDMF (Smooth muscle cell-derived migration factor), Tachykinins, TGF-beta, and Thrombospondin.

Factors modulating growth, chemotactic behavior, and/or functional activities of vascular endothelial cells include aFGF, ANF, Angiogenin, Angiostatin, Angiotropin, AtT20-ECGF, B61, bFGF, bFGF inducing activity, CAM-RF, ChDI, CLAF, Cox-2, ECG, ECI, EDM, EGF, EMAP, Neurothelin, Endostatin, Endothelial cell growth inhibitor, Endothelial cell-viability maintaining factor, Ephrins, Epo, FGF-5, IGF-2, HBNF, HGF, HUAF, IFN-gamma, IL1, K-FGF, LIF, MD-ECI, MECIF, MMPs, NO, Oncostatin M, PAI-2, PD-ECGF, PDGF, PF4, PlGF, Prolactin, TIMPs, TNF-alpha, TNF-beta, Transferrin, urokinase, and VEGI. Proteins that have angiogenesis activity in vivo include fibroblast growth factors, Angiogenin, Angiopoietin-1, EGF, HGF, VEGA, VEGB, VEGC, VEGD, VEGE, VEGF, TNF-alpha, TGF-beta, PD-ECGF, PDGF, IGF, IL8, and Growth hormone. Fibrin fragment E also has angiogenic activity. In addition, Angiopoietin-1 plays a prominent role in vasculogenic and angiogenic processes. PF4 and a 16 kDa fragment of Prolactin are inhibitory in vivo.

Angiogenic activities also include a number of other compounds such as prostaglandins E1 and E2, steroids, heparin, 1-butyryl glycerol (monobutyrin) secreted by adipocytes, and many undefined derivatives of the arachidonic acid metabolism. The biologically active principle extracted from some carcinoma cells and identified as nicotinic amide is also a potent angiogenic compound in several bioassays.

Signal Transduction Inhibitors

In addition to Herceptin, there are numerous other signal transduction inhibitors that can be used in the methods of the present invention to prevent or treat cancer or angiogenesis related diseases. Examples of other signal transduction inhibitors include ^{Pr}Gleevec™ (also called ST1571), IMC-C225 (from ImClone), ZD 1839 (from AstraZeneca), IGF-1, and platelet-factor 4. Other exemplary signal transduction inhibitory therapies include anti-estrogen therapies such as an adreno-ovariectomy surgical procedure and compounds that inhibit estrogen activity (e.g., tamoxifen or an LHRH analog). Still other signal transduction inhibitory therapies include anti-androgen therapies such as surgical castration and compounds that inhibit androgen activity (e.g., ˜50 mg daily bicalutamide, cyproterone acetate, dexamethasone, or an LHRH analog).

Anti-angiogenesis tyrosine kinase inhibitors can also be used to modulate tumor cells that have receptors for these kinases. For example, SU6668 can be used to inhibit PDGF, FGF, and VGEF. SU5416 and ZD4190 inhibit VGEF. Additionally, IMC-IC11 (DC101, from ImClone) is an anti-VGEF antibody that inhibits VGEF. Other exemplary angiogenesis inhibitors are listed in FIGS. 2A-2G.

Other Compounds for Inclusion in Individual or Combination Therapies

In general, additional drugs for the treatment of cancer or angiogenesis related diseases may be identified from large libraries of both natural product or synthetic (or semi-synthetic) extracts or chemical libraries according to methods known in the art. Those skilled in the field or drug discovery and development will understand that the precise source of test extracts or compounds is not critical to the methods of the invention.

Accordingly, virtually any number of chemical extracts or compounds can be screened for their effect on the activity or expression of cancer or angiogenesis related factors. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

When a crude extract is found to modulate the activity or expression of a cancer or angiogenesis related factor, further fractionation of the positive lead extract is necessary to isolate chemical constituent responsible for the observed effect. Thus, the goal of the extraction, fractionation, and purification process is the careful characterization and identification of a chemical entity within the crude extract. Methods of fractionation and purification of such heterogeneous extracts are known in the art. If desired, compounds shown to be useful agents for the treatment of cancer or an angiogenesis related disease are chemically modified according to methods known in the art. Compounds identified as being of therapeutic value are subsequently analyzed using any standard animal model of angiogenesis or cancer known in the art.

Assays and Animal Models for the Testing of Therapies of the Invention

If desired, the therapies of the invention can be tested for their effect on cancer and angiogenesis using the SCID mouse model described herein. Additionally, there are numerous standard assays and animal models that can be used to determine the efficacy of particular therapies for inhibiting angiogenesis [Auerbach et al, Dev. Biology 41: 391-394 (1974); Brown et al, Laboratory Investigation 75(4): 539-555 (1996); Castellot et al., Journal of Cellular Physiology 127: 323-329 (1986); Gaudric et al, Research 24: 181-188 (1992); Greenblatt and Shubik, Journal of the National Cancer Institute 41: 111-124 (1968); Hayek et al., Microvasc. Research 41: 203-209 (1991); Lichtenberg et al, Pharmacol Toxicology 84(1): 34-40 (1999); Lichtenberg et al, Pharmacol Toxicology 81(6): 280-284 (1997); Mourad et al., British Journal of Dermatology 123: 21-28 (1990); Nissanov et al, Laboratory Investigation 73(5): 734-739 (1995); Okamura et al, Biochemical and Biophysical Research Communications 186: 1471-1479 (1992); Olivo et al., Anat. Rec. 234: 105-115 (1992); O'Reilly et at, Cell 79(2): 315-328 (1994); O'Reilly et al, Cell 88: 277-285 (1997); Passaniti et al., Lab. Invest. 67: 519-528 (1992); Peek et al, Experimental Pathology 34: 35-40 (1988); Polyerini et al, Methods in Enzymology 198; 440-450 (1991); Ribatti et al., Journal of Vascular Research 34(6): 455-463 (1997); Sato et al, Journal of Investigative Dermatology 95: 85S-89S (1990); Wilting J et al, Anat. Embryology 183: 259-271 (1991)]. The individual and combination therapies can also be tested in standard human clinical trials.

Administration of Therapies

A therapy of the invention may be administered to humans, domestic pets, livestock, or other animals with a pharmaceutically acceptable diluent, carrier, or excipient, in unit dosage form.

The compounds optionally may be administered as pharmaceutically acceptable salts, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

The chemical compounds for use in such therapies may be produced and isolated as described herein or by any standard technique known to those in the field of medicinal chemistry. Conventional pharmaceutical practice may be employed to provide suitable formulations or compositions to administer the identified compound to patients suffering from cancer or an angiogenesis related disease or at increased for cancer or an angiogenesis related disease. Administration may begin before or after the patient is symptomatic.

Any appropriate route of administration may be employed. For example, the therapy may be administered either directly to the tumor (for example, by injection) or systemically (for example, by any conventional administration technique). Preferably, the therapy is administered using a controlled-release microchip, microparticle extended-release formulation, polymeric nanoparticle, or transdermal delivery system (as described, for example, in LaVan et al., Nature Reviews 1:77-84, 2000 or Santini et al., Nature 397:335-338, 1999). Administration of the compounds may also be oral, topical parenteral, intravenous, intraarterial, subcutaneous, intramuscular, intracranial, intraorbital, ophthalmalic, intraventricular, intracapsular, intraspinal, intracisternal, intraperitoneal, or intranasal. Alternatively, the compounds may be administered as part of a suppository. Therapeutic formulations may be in the form of liquid solutions or suspensions; for oral administration, formulations may be in the form of tablets or capsules; and for intranasal formulations, in the form of powders, nasal drops, or aerosols. The compounds in a combination therapy may be administered simultaneously or sequentially. For example, one or more compounds in a combination therapy can be administered until the compound(s) normalize the blood vessel network of the tumor and thereby increase the accessibility of the tumor to other therapeutic agents, and then one or more additional compounds can be administered instead of, or in addition to, the originally administered compound(s). The dosage of the therapeutic compounds in a pharmaceutically acceptable formulation depends on a number of factors, including the size and health of the individual patient. The dosage to deliver may be determined by one skilled in the art. For example, compounds that are administered as part of a combination therapy of the invention are typically administered at a dose equal to or at least 25, 50, or 75% lower than the corresponding dose for the compound when it is used individually. An exemplary dosing regimen for Herceptin includes a 4 mg/kg loading dose followed by a weekly dose of 2 mg/kg. Other suggested doses include 200-400 or 400-600 mg/body/day for ^{Pr}Gleevec™, a 400 mg/m² loading dose followed by a weekly dose of 250 mg/m² for IMC-C225, and 150-1000 mg/body/day for ZD1839.

Methods well known in the art for making formulations are found, for example, in “Remington: The Science and Practice of Pharmacy” ((19th ed.) ed. A. R. Gennaro AR., 1995, Mack Publishing Company, Easton, Pa.). Formulations for parenteral administration may, for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

If desired, treatment with a compound identified according to the methods described above, may be combined with more traditional therapies for cancer or an angiogenesis related disease (e.g., cytotoxic agents, radiation therapy, or surgical removal of cancerous cells).

### Other Embodiments

From the foregoing description, it will be apparent that variations and modifications may be made to the invention described herein to adopt it to various usages and conditions. Such embodiments are also within the scope of the following claims.

All publications, patent applications, and patents mentioned in this specification are herein incorporated by reference to the same extent as if each independent publication, patent, or patent application was specifically and individually indicated to be incorporated by reference.

What is claimed is:

1. A method for selecting a combination therapy for the treatment, stabilization, or prevention of a cancer or an angiogenesis related disease in a mammal, said method comprising the steps of:
(a) analyzing the expression profile of more than one mRNA and/or protein in a sample obtained from said mammal; and
(b) selecting a therapy that comprises two or more compounds that each (i) decrease the expression level or activity of an mRNA or protein that has a higher than normal expression level in said mammal and/or (ii) increase the expression level or activity of an mRNA or protein that has a lower than normal expression level in said mammal.

2. A method for preventing, delaying, or treating a cancer or an angiogenesis related disease in a mammal, said method comprising the steps of:
(a) analyzing the expression profile of more than one mRNA and/or protein in a sample obtained from said mammal;
(b) selecting a therapy that comprises two or more compounds that each (i) decrease the expression level or activity of an mRNA or protein that has a higher than normal expression level in said mammal and/or (ii) increase the expression level or activity of an mRNA or protein that has a lower than normal expression level in said mammal; and
(c) administering said therapy to said mammal in an amount sufficient to treat, stabilize, or prevent said cancer or angiogenesis related disease.

3. A method for stratification of subjects involved in a clinical trial of a combination therapy comprising two or more compounds for the treatment, stabilization, or prevention of a cancer or an angiogenesis related disease in a mammal, said method comprising the steps of:
(a) analyzing the expression profile of more than one mRNA and/or protein in a sample obtained from a subject; and
(b) determining the presence of a lower or higher than normal expression level for more than one mRNA and/or protein in said sample before, during, or after said clinical trial, wherein the presence of said expression profile in said subject places said subject into a subgroup for said clinical trial.

4. The method of claim 1, wherein said therapy comprises a signal transduction inhibitor.

5. The method of claim 4, wherein all of the pharmaceutically active compounds in said therapy are signal transduction inhibitors.

6. The method of claim 4, wherein said therapy comprises a compound other than a signal transduction inhibitor.

7. A method for selecting a signal transduction inhibitor for the treatment, stabilization, or prevention of a cancer or an angiogenesis related disease in a mammal, said method comprising the steps of:
(a) analyzing the expression profile of one or more mRNA molecules and/or proteins in a sample obtained from said mammal, wherein the expression or activity of said mRNA molecule or protein can be modulated by a signal transduction inhibitor; and
(b) selecting a signal transduction inhibitor that (i) decreases the expression level or activity of an mRNA or protein that has a higher than normal expression level in said mammal and/or (ii) increases the expression level or activity of an mRNA or protein that has a lower than normal expression level in said mammal.

8. A method for preventing, delaying, or treating a cancer or an angiogenesis related disease in a mammal, said method comprising the steps of:
(a) analyzing the expression profile of one or more mRNA molecules and/or proteins in a sample obtained from said mammal, wherein the expression or activity of said mRNA molecule or protein can be modulated by a signal transduction inhibitor;
(b) selecting a signal transduction inhibitor that (i) decreases the expression level or activity of an mRNA or protein that has a higher than normal expression level in said mammal and/or (ii) increases the expression level or activity of an mRNA or protein that has a lower than normal expression level in said mammal; and
(c) administering said signal transduction inhibitor to said mammal in an amount sufficient to treat, stabilize, or prevent said cancer or angiogenesis related disease.

9. A method for stratification of subjects involved in a clinical trial of a signal transduction inhibitor for the treatment, stabilization, or prevention of a cancer or an angiogenesis related disease in a mammal, said method comprising the steps of:
(a) analyzing the expression profile of one or more mRNA molecules and/or proteins in a sample obtained from a subject, wherein the expression or activity of said mRNA molecule or protein can be modulated by a signal transduction inhibitor; and
(b) determining the presence of a lower or higher than normal expression level for one or more mRNA molecules and/or proteins in said sample before, during, or after said clinical trial, wherein the presence of said expression profile in said subject places said subject into a subgroup for said clinical trial.

10. The method of claim 7 or 8, further comprising selecting another signal transduction inhibitor that (i) decreases the expression level or activity of another mRNA or protein that has a higher than normal expression level in said mammal and/or (ii) increases the expression level or activity of another mRNA or protein that has a lower than normal expression level in said mammal.

11. The method of claim 7 or 8, further comprising repeating step (b) until signal transduction inhibitors are selected for the modulation of the expression or activity of all the cancer or angiogenesis related mRNA molecules or proteins that have lower or higher than normal expression levels in said mammal.

12. The method of claim 4 or 7, wherein said signal transduction inhibitor is Herceptin.

13. The method of claim 4 or 7, wherein said signal transduction inhibitor is ^{Pr}Gleevec™.

14. The method of claim 1 wherein said therapy comprises five compounds.

15. The method of claim 1 or 7, wherein step (a) comprises comparing the expression level of an mRNA or protein in said sample to the corresponding level in a control sample.

16. (canceled)

17. The method of claim 1 or 7, wherein at least one of said compounds or said signal transduction inhibitors modulates the expression of at least 3 mRNA molecules or proteins.

18. (canceled)

19. The method of claim 1 or 7, wherein the expression profile comprises the expression level of mRNA molecules or proteins selected from the group consisting of vascular endothelial growth factor, transforming growth factor alpha, angiopoietin-1, plasminogen activator inhibitor-1, and thrombospondin-1.

20. (canceled)

21. (canceled)

22. (canceled)

23. The method of claim 1 or 7, wherein said therapy inhibits angiogenesis of said cancer by at least 25%.

24. (canceled)

25. The method of claim 1 or 7, wherein said mammal or said subject is a human.

26. (canceled)

27. (canceled)
